(19) United States
(12) Patent Application Publication
(10) Pub. No.: US 2009/0107857 A1
(43) Pub. Date: Apr. 30, 2009

(54) TOOTHBRUSH HOLDER

(76) Inventors: Jason Alexander Fretwell, Winchester (GB); Kurt Andrew John Schramm, London (GB)

Correspondence Address: HOFFMAN WARNICK LLC, 75 STATE STREET, 14TH FLOOR, ALBANY, NY, 12207, US

(21) Appl. No.: 12/255,073

(22) Filed: Oct. 21, 2008

Related U.S. Application Data
(63) Continuation of application No. 11/276,030, filed on Feb. 10, 2006, now abandoned.

(30) Foreign Application Priority Data
Aug. 11, 2003 (GB) 0318816.6
Aug. 11, 2004 (GB) PCT/GB2004/003468

(51) Int. Cl. A45D 44/18

(52) U.S. Cl. 206/209.1

(57) ABSTRACT

A toothbrush holder comprises a container for containing a liquid and at least the head of a toothbrush, the container having an opening through which the head of the toothbrush may pass into the container and a closing member for intimately surrounding at least a portion of the handle of the toothbrush and closing the open end of the container when the head of the toothbrush is received within the container.

### CLAIM FOR PRIORITY

This is a continuation of the currently pending U.S. patent application having Ser. No. 11/276,030, filed on Feb. 10, 2006, which claims priority to International Application PCT/GB2004/003468, with an International Filing Date of Aug. 11, 2004, which claims the benefit of United Kingdom patent application No. 0318816.6, filed Aug. 11, 2003. Each of the above-mentioned patent applications is hereby incorporated by reference herein.

### FIELD OF THE INVENTION

The present invention relates a toothbrush holder, and in particular to a toothbrush holder in which the head of the toothbrush is held in a disinfectant, sterilising or antibacterial liquid.

Conventionally, when a toothbrush is not being used, the head of the toothbrush is left exposed to the environment. This may occur when the toothbrush is either placed on a surface, or held in rack-type holder. While the head of the toothbrush is exposed to the environment, the toothbrush may become contaminated. When the toothbrush is next used, any germs or other contamination on the head of the toothbrush will be transferred to the mouth of the user.

A toothbrush holder has previously been proposed in which a number of toothbrushes are each received within a compartment of a toothbrush holder including a sanitising liquid. An example of such a toothbrush holder is disclosed in U.S. Pat. No. 4,473,152. In this arrangement, the compartments containing the sterilising liquid have an upper opening, allowing the head of the toothbrush to be passed through the open top of the container into the sterilising liquid. The toothbrush holder is intended to be free standing or wall mounted.

According to the present invention, a toothbrush holder comprises a container for containing a liquid and at least the head of a toothbrush, the container having an opening through which the head of the toothbrush may pass into the container, and a closing member for intimately surrounding at least a portion of the handle of the toothbrush and closing the open end of the container when the head of the toothbrush is received within the container.

With the toothbrush holder according to the present invention, the open end of the container containing the liquid is closed around the handle of the toothbrush by the closing member. This both prevents spillage of the liquid from the holder, and prevents contamination of the liquid within the whole. Also, this allows the container to be inverted. This is of particular advantage where the toothbrush is able to stand on its end opposite the head. This is common for electric toothbrushes in which the handle contains batteries to power the toothbrush, and is therefore much larger and heavier than the head end of the toothbrush.

It will be appreciated that the position of the closing member on the handle of the toothbrush will be dependent upon the length of the head of the toothbrush, the length of the handle and the depth of the container. It will therefore be appreciated that reference to “handle” of the toothbrush is a reference to any part of the toothbrush from the head to the end of the toothbrush, and is not limited to the part of the toothbrush actually held by the user during use.

It is preferred that the closing member hermetically seals the open end of the container. In this condition, the toothbrush can be transported with the head of the toothbrush contained within the liquid within the holder. This allows the toothbrush to be carried in a wash bag or the like with the head of the toothbrush retained the within the liquid, and without the risk that the liquid spills in transit.

In one example, the closing member may be integrally formed or fixed on the handle portion of the toothbrush, and arranged so that insertion of the head of the toothbrush into the container brings the closing member into contact with the open end of the container to close this. In this case, it is important that the closing member is of such a size, or is positioned on the handle of the toothbrush in such a position that this does not interfere with the use of the toothbrush when cleaning teeth.

In a preferred example however, the closing member is in the form of a collar that is removably attached to the handle of the toothbrush. In this way, the closing member can be removed from the toothbrush whilst this is being used, and therefore does not interfere with the user when cleaning their teeth. When the toothbrush is to be stored, the collar can be mounted on or around the handle of the toothbrush, being arranged to come into contact with and close the open end of the container when the head of the toothbrush is positioned within the container.

When the closing member is releasably attachable to the handle of the toothbrush, it is possible for the closing member to be clipped, snapped or otherwise attached to the handle of the toothbrush to retain the closing member on the handle of the toothbrush whilst allowing its removal when required. Alternatively, the closing member may be provided on or around the handle of the toothbrush and arranged to be held in position by engagement or interaction of the closing member with the container. In one example, the closing member is arranged to be received within the open end of the container, in which case the inside of the container wall near the opening acts to hold the closing member around the handle of the toothbrush. In another example, the closing member is arranged to engage the rim of the open end of the container, for example clipping into the rim, to hold this in place around the handle of the toothbrush. Alternatively, the closing means may be held in place by a clip or separate connector holding the closing member around the handle of the toothbrush.

Advantageously, the container is formed from a plastic material. Preferably the container is transparent or translucent, allowing a user to see the contents of the container. The containers can be made in various colours, which can be useful in identifying different toothbrushes, for example the toothbrushes for a number of different people using the same bathroom.

The container advantageously includes a base having a larger area then the main body of the container, giving the container greater stability. Alternatively or additionally, the container may include a weighted portion or may be formed from dense material, to increase the stability of the container. This is important when the holder is stood on a surface with the toothbrush standing generally vertically above the container. Advantageously, sealing members are provided to create a seal between the closing member and the open end of the container and/or between the handle of the toothbrush and the closing member.

The liquid contained within the container is preferably a disinfectant, sterilising, antibacterial or sterile liquid. Where a sterile liquid is used, it is able to prevent further contamination of the head of the toothbrush. Where a disinfectant, sterilising or antibacterial liquid is used, this is able to assist in killing bacteria, germs or other contamination already on the head of the toothbrush.

An example of the present invention will now be described, with a reference to the accompanying drawings in which:

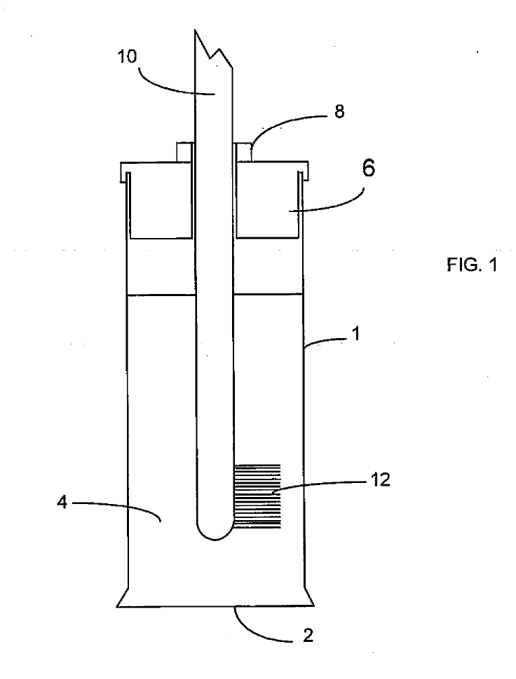

FIG. 1 shows a cross-sectional view of a container including a toothbrush and closing member;

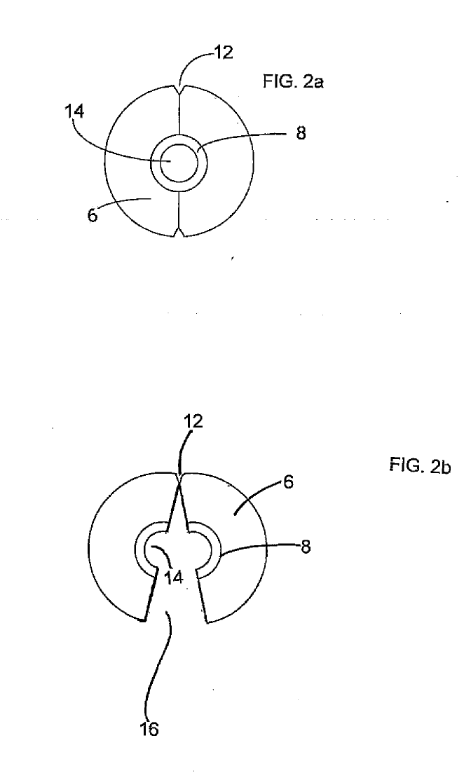

FIGS. 2a and 2b show plain views of a closing member; and,

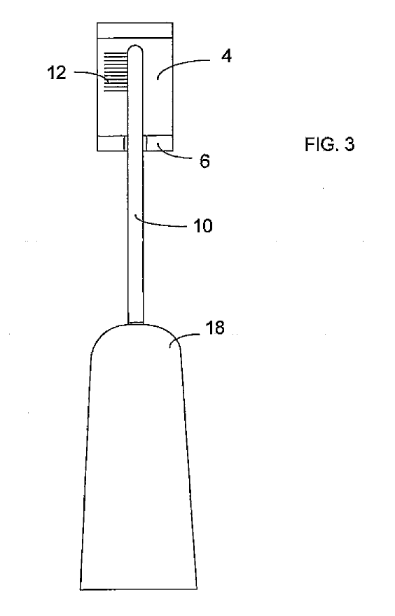

FIG. 3 shows an example of a holder used with an electric toothbrush.

As shown in FIG. 1, a container 1 is provided having a base 2 and a liquid receiving chamber. The container is open at the top end, opposite the base 2. The container 1 is arranged to include a sterilising, disinfectant, antibacterial or sterile liquid 4. The container 1 is made from a coloured, translucent, plastics material. The base 2 of the container 1 has a larger diameter then the main body of the container 1 is made from a dense plastic material to give the container stability when standing on its base. Closing member 6 includes two portions that are hingably connected together. When the two portions are closed, as shown in FIG. 2a, an opening 14 is defined between the portions for receiving the handle 10 of a toothbrush. When the two parts of the closing member are hinged apart, as shown in FIG. 2b, the closing member can be positioned around the handle 10 of a toothbrush.

The closing member includes a seal 8, which may be formed from a suitable material such as rubber or foam, which seals against the handle 10 of the toothbrush as the closing member is closed around the handle 10 of the toothbrush.

In the example shown in FIG. 1, the toothbrush is positioned within the container 1 so that the head 12 of the toothbrush is received within the liquid 4. In this position, the closing member 6 is received within the open end of the container 1 which holds the closing member 6 around the handle 10 of the toothbrush. In this way, the open end of the container 1 is close to retaining the head 12 of the toothbrush in the liquid 4 within the container 1.

The closing member 6 may be formed from a resilient material to ensure that a hermetic seal is provided to close the open end of the container 1. Alternatively, a sealing means may be provided to give the desired hermetic seal, for example in the form of an O-ring around the open end of the container 1. In some cases, a hermetic seal may not be required, for example where the holder is intended to remain within a bathroom, and therefore there is limited likelihood of liquid being spilt from the container, and where the container is not used in an inverted condition.

In an alternative embodiment, the closing member 6 is in the form of a collar, which is fixed to the handle 10 of the toothbrush without requiring any interaction between the closing member and the end of the container 1. This may be achieved by forming the parts of the closing member 6 with a clip to connect the two parts of the closing member opposite to the hinged side. In this way, after the closing member is provided to encircle the handle of the toothbrush, the closing member can be holding place by clipping the two parts together. Alternatively, a separate band or other closure could be provided to hold the parts of the closing member together around the handle of the toothbrush. In this case, it will not be necessary for the two parts of the closing member to be connected.

In another example of the present invention, the closing member may be permanently fixed to the handle of the toothbrush. The closing member could be a separate member from the handle of the toothbrush fixed to the toothbrush handle, for example by adhesive and welding. Alternatively, the closing member could be formed integrally with the handle of the toothbrush.

The liquid contained within the container may be a sterile liquid, such as sterile water, which prevents further contamination of the head 12 of the toothbrush. However, it is preferred that the liquid is a weak disinfecting, sterilising or antibacterial solution. This helps to kill any germs or bacteria on the head 12 of the toothbrush.

As shown in FIG. 3, the container 1 may be provided to contain the liquid 4 and the head 12 of the toothbrush in an inverted condition. In the example shown, the toothbrush is an electric toothbrush having a main body 18 including the power and the motor for the operation of the toothbrush, and a stem or handle portion 18 on which the head 12 is provided. In this case, the toothbrush is stored standing on the base of the body 18, with the holder held on the head 12 by the closing means 6.

What is claimed is:

1. A toothbrush holder comprising:
a container for containing a liquid and at least the head of a toothbrush, the container having an opening through which the head of the toothbrush may pass into the container; and,
a closing member for intimately surrounding at least a portion of the handle of the toothbrush and closing the open end of the container when the head of the toothbrush is received within the container.

2. A toothbrush holder according to claim 1, in which the closing member hermetically seals the open end of the container.

3. A toothbrush holder according to claim 1, in which the closing member is arranged so that insertion of the head of the toothbrush into the container brings the closing member into contact with the open end of the container to close this.

4. A toothbrush holder according to claim 1, in which the closing member is integrally formed with the handle of the toothbrush.

5. A toothbrush holder according to claim 1, in which the closing member is permanently connected to the handle of the toothbrush, for example by welding, adhesive or the like.

6. A toothbrush holder according to claim 1, in which the closing member is in the form of a collar that is removably attached to the handle of the toothbrush.

7. A toothbrush holder according to claim 6, in which the closing member is clipped, snapped or otherwise attached to the handle of the toothbrush to retain the closing member on the handle of the toothbrush whilst allowing its removal when required.

8. A toothbrush holder according to claim 6, in which the closing member is held in position on the handle of the toothbrush by engagement or interaction of the closing member with the container.

9. A toothbrush holder according to claim 8, in which the closing member is arranged to be received within the open end of the container, in which case the inside of the container wall near the opening acts to hold the closing member around the handle of the toothbrush.

10. A toothbrush holder according to claim 8, in which the closing member is arranged to engage the rim of the open end of the container, for example clipping into the rim, to hold this in place around the handle of the toothbrush.

11. A toothbrush holder according to claim 6, in which the closing member is held in place by a clip or separate connector holding the closing member around the handle of the toothbrush.

12. A toothbrush holder according to claim 1, in which the container is formed from a plastic material.

13. A toothbrush holder according to claim 1, in which the container includes a base having a larger area then the main body of the container, giving the container greater stability.

14. A toothbrush holder according to claim 1, in which sealing members are provided to create a seal between the closing member and the open end of the container and/or between the handle of the toothbrush and the closing member.

15. A toothbrush holder according to claim 1, in which the liquid contained within the container is a disinfectant, sterilising, antibacterial or sterile liquid.
